(19) United States
(12) Patent Application Publication
(10) Pub. No.: US 2020/0058125 A1
(43) Pub. Date: Feb. 20, 2020

(54) Comparative cancer survival models to assist physicians to choose optimal treatment

(71) Applicant: Mikhail Teverovskiy, Stamford, CT (US)

(72) Inventor: Mikhail Teverovskiy, Stamford, CT (US)

(21) Appl. No.: 15/998,481

(22) Filed: Aug. 14, 2018

(51) Int. Cl. G06T 7/00; G06T 7/11; G06T 7/194; G16H 50/30; G16H 50/50

(52) U.S. Cl. CPC G06T 7/0016; G06T 7/11; G06T 7/194; G06T 2207/30028; G16H 50/50; G06T 2207/30024; G16H 50/30

(57) ABSTRACT

A computer implemented method and a system choosing optimal disease treatment among several possible treatment options for a patient are provided. The system computes cancer-free survival rates for each considered treatment based on predicting recurrence rate of a disease and/or cancer outcome for a particular patient. The treatment survival models use quantitative data from histopathological images of the patient, clinical data and other patient information. The system segments the histopathological images into biologically meaningful components; automatically determines disease-affected regions in one or more of the segmented image components. The system also partitions the disease-affected regions in each image into a number clusters. Those that are determined to be the most associated with the disease outcome are used as a source of the imaging information for the survival modeling. Optimal treatment is suggested as the treatment with probability of the cancer free survival within a certain time period is maximized.

### BACKGROUND

Colon cancer is a disease that originates in a large intestine or a rectum and affects the lives of men and women. Colon cancer often results in death if it remains undiagnosed, recurs, or spreads throughout a patient's body. The probability of a disease free survival of cancer patients within a time period of 5 years following complete surgical resection of all cancerous tissues is one of the predictive factors for estimating recurrence of colon cancer in cancer patients. Despite the generally good outcomes associated with early stage colon cancer treatments, a significant number of patients are still prone to disease recurrence and ultimately die from the disease.

An immediate step after cancer diagnosis is treatment planning. The goal of treatment planning is to choose a set of medical procedures comprising, for example, surgery, radiation, chemotherapy, etc., aiming to completely or partially cure the disease in such way that a patient's life is saved or maximally extended. Often more than few treatment options are applicable to similar disease conditions such as in the case of cancer. The decision of choosing a treatment plan for a patient is usually based on several components comprising, for example, clinical information, available technology such as curative devices for treatment of a disease, financial information such as cost of treatment, quality of life for a patient during and/or after a treatment, a professional such as a doctor's experience and specialty, etc. While medical factors and personal factors are always taken in consideration as a basis for medical decisions regarding choice of a treatment plan, other factors such as magnetic resonance imaging (MRI) reports, computed tomography (CT) scans, etc., that are equally helpful for choosing a treatment plan, are not considered at all. There is a need for a computer implemented method and system that recommends a treatment plan for a patient based on medical factors, personal factors, and other diagnostic factors.

Treatment success can be quantified by a survival rate, which is defined as a probability of disease free survival of a patient within a time span of, for example, 5 years, 7 years, 10 years, etc., after a treatment is completed. One of the predictive factors for cancer patients is survival rate. The predictive tools used today for predicting a patient's survival are based on tumor-nodes-metastasis (TNM) cancer staging system, which is maintained by the American Joint Committee on Cancer (AJCC). These predictive tools are implemented in the form of tables and/or nomograms, where patients are grouped using the TNM cancer staging system. The standard TNM cancer staging system cannot predict which patient's medical condition can recur and/or needs additional therapy. Moreover, in the case of colon cancer patients, the conventional TNM cancer staging system does not provide variable prognoses for early stage JIB colon cancer patients. Predictive tables are widely validated since they are relatively easy to use. In the TNM cancer staging system, a likelihood of cancer free survival is determined by a location of a particular patient's profile in a table bin. However, stratification of patients into discrete categories fails to recognize a heterogeneous nature of cancer outcomes within each category, and therefore results in inaccurate personalized predictions. There is a need for a patient survival prediction system that predicts a survival rate and/or a recurrence rate of a disease for a patient considering heterogeneous nature of cancer outcomes in all patients.

Nomograms are statistical tools that estimate probability of cancer free survival. Unlike probability tables, where predictors are collapsed into discrete bins, nomograms incorporate continuous variables into a prognostic score to quantify the risk of disease recurrence. In the case of nomograms, original values of the predictors are preserved, and lead to an improved accuracy of risk estimation for disease recurrence in a patient. However, the main disadvantage of the nomogram approach is that the effect of the predictors on quantification of risk of disease recurrence is measured by a pathologist based upon his/her subjective evaluation. Therefore, the reproducibility is limited.

Efficient image quantitation requires segmentation of histopathological images. Major challenges in segmentation of histopathological images are large intensity variations and pixel noise. Some approaches addressing these challenges use either substantial learning schemes or time consuming semi-supervised algorithms. However, the method of image segmentation has not been used yet to predict cancer treatment outcomes. There is a need for a patient survival prediction system that uses image segmentation for predicting cancer treatment outcomes.

Hence, there is a long felt but unresolved need for a computer implemented method and system that predicts recurrence of a disease in a patient and a treatment outcome for the patient. Moreover, there is a need for a computer implemented method and system that generates one or more treatment plans for a patient based on medical factors, personal factors, and other diagnostic factors. Furthermore, there is a need for a computer implemented method and system that predicts a survival rate and/or a recurrence rate of a disease for a patient while considering the heterogeneous nature of disease outcomes in all patients categorized in different classes of the disease type. Moreover, there is a need for a computer implemented method and system that uses image segmentation for predicting disease treatment outcomes.

### SUMMARY OF THE INVENTION

The computer implemented method and the disease recurrence prediction system disclosed herein address the above stated needs for predicting recurrence of a disease in a patient and a treatment outcome for the patient. Moreover, the computer implemented method and the disease recurrence prediction system disclosed herein generate one or more treatment plans for a patient based on medical factors, personal factors, and other diagnostic factors. Furthermore, the computer implemented method and the disease recurrence prediction system disclosed herein predict a survival rate and/or a recurrence rate of a disease for a patient while considering a heterogeneous nature of disease outcomes in all patients categorized in different classes of the disease type. Moreover, the computer implemented method and the disease recurrence prediction system disclosed herein use image segmentation for predicting disease treatment outcomes.

The computer implemented method disclosed herein employs the disease recurrence prediction system comprising at least one processor configured to execute computer program instructions for predicting recurrence of a disease in a patient and a treatment outcome for the patient. The disease recurrence prediction system receives multiple histopathological images and patient information from multiple sources. The patient information comprises, for example, the patient's clinical information, demographic information, imaging information, etc. The disease recurrence prediction system segments the histopathological images to generate image components of the histopathological images. During segmentation, the disease recurrence prediction system segments background image components from tissue image components of the histopathological images. The disease recurrence prediction system then segments white space image components from stromal-epithelium image components of the tissue image components of the histopathological images. The disease recurrence prediction system then segments stromal image components from epithelium image components of the stromal-epithelium image components of the histopathological images.

The disease recurrence prediction system determines disease affected regions in one or more of the image components of the histopathological images, for example, by performing a spatial analysis of the image components of the histopathological images. In an embodiment, the spatial analysis comprises performing an iterative expansion of one of the image components of the histopathological images. The disease recurrence prediction system partitions the determined disease affected regions in the image components of the histopathological images into multiple clusters, for example, by performing a texture based segmentation of the image components of the histopathological images. The disease recurrence prediction system quantitates the clusters of the determined disease affected regions in the image components of the histopathological images based on multiple measurement parameters. The measurement parameters comprise, for example, area, perimeter, color, fractal dimensions of region boundaries, texture features, etc. The disease recurrence prediction system determines one or more key clusters as the most associated with a heterogeneous nature of a disease outcome from the quantitated clusters of the determined disease affected regions. The disease recurrence prediction system then quantitates the determined key clusters based on the measurement parameters.

The disease recurrence prediction system predicts the recurrence of the disease in the patient and the treatment outcome for the patient via statistical modeling of the patient's survival based on the quantitation of the key clusters and the patient information. In an embodiment, the disease recurrence prediction system performs the statistical modeling of the patient's survival based on the quantitation of the key clusters and the patient information at a time instant before the treatment of the patient and/or a time instant after the treatment of the patient. In an embodiment, the disease recurrence prediction system generates one or more treatment plans for the patient based on the statistical modeling of the patient's survival. In this embodiment, the disease recurrence prediction system predicts a probability of a disease free survival of the patient within a time period for each of the generated treatment plans.

### BRIEF DESCRIPTION OF THE DRAWINGS

FIG. 1 illustrates a computer implemented method for predicting recurrence of a disease in a patient and a treatment outcome for the patient.

FIG. 2 exemplarily illustrates a histopathological image of a colon tissue.

FIG. 3 exemplarily illustrates multiple image components of the histopathological image of the colon tissue generated by segmentation of the histopathological image by a disease recurrence prediction system.

FIG. 4A exemplarily illustrates a histopathological image of the colon tissue with cancer clusters.

FIG. 4B exemplarily illustrates a histopathological image of the colon tissue with relabeled cancer clusters.

FIG. 5 exemplarily illustrates a graphical representation of an estimation of a disease free survival of multiple early stage colon cancer patients over a period of time, performed by the disease recurrence prediction system.

### DETAILED DESCRIPTION OF THE INVENTION

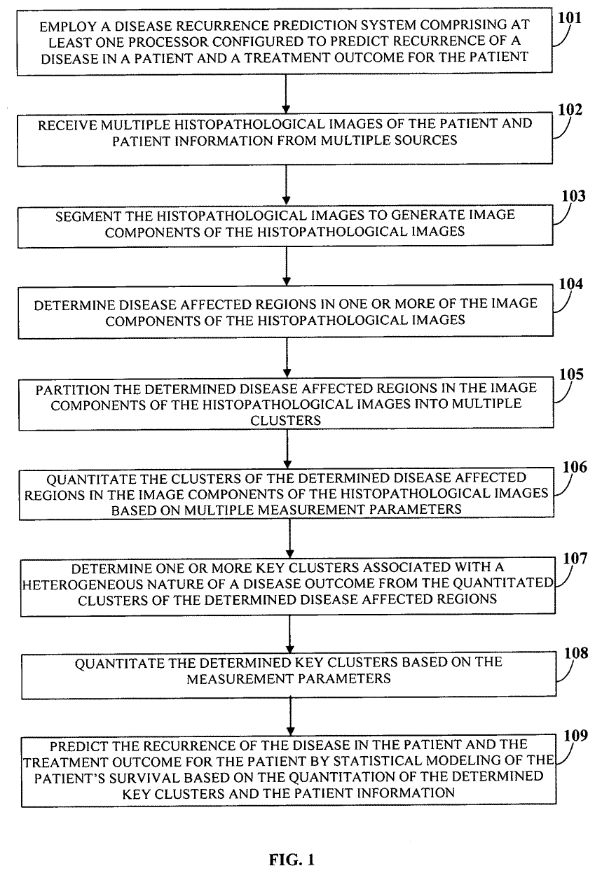

FIG. 1 illustrates a computer implemented method for predicting recurrence of a disease in a patient and a treatment outcome for the patient. The computer implemented method disclosed herein employs 101 a disease recurrence prediction system comprising at least one processor configured to execute computer program instructions for predicting recurrence of a disease in a patient and a treatment outcome for the patient. The disease recurrence prediction system is implemented as a web based platform with a graphical user interface (GUI) for data input and treatment simulations. The web based platform is accessible by multiple users, for example, qualified users such as medical doctors, pathologists, clinicians, etc., via a network. The users can upload the patient's information into the disease recurrence prediction system using configurable templates via the GUI of the disease recurrence prediction system. The disease recurrence prediction system provides the users with a computational tool based on quantitative analyses of histopathological images and a computerized approach to accurately predict disease recurrences. The disease recurrence prediction system is based on advanced predictive algorithms that perform survival prediction for a patient using tumor-node-metastasis (TNM) classification factors and non-TNM classification factors.

The disease recurrence prediction system receives 102 multiple histopathological images of a patient and patient information from multiple sources. The patient information comprises, for example, the patient's clinical information such as information associated with prior diagnosis of a patient' disease such as pathological tumor stage, tumor size, tumor aggressiveness grade such as Gleason score for prostate cancer, depth of tumor invasion, or number of lymph nodes examined, biomarker expressions, genetic markers, etc., demographic information such as age, gender, race, sex, etc., imaging information such as quantitative features extracted from digital images that represent the tumor and acquired under a microscope, magnetic resonance imaging (MRI) information, X-ray computed tomography (CT) information, ultrasound information, etc. For example, the disease recurrence prediction system uses quantitative tumor characteristics extracted from digitized histopathological images of colon tissues for performed advanced predictions. The sources comprise, for example, haematoxylin and eosin (H&E) stained slides of tissues received from pathologists, histopathological images converted from the H&E stained slides of tissues by the ScanScope® digital slide scanner of the Aperio Technologies, Inc., etc.

The disease recurrence prediction system integrates tumor-node-metastasis (TNM) classification factors with non-TNM classification factors comprising, for example, histopathological images, other clinical information associated with a patient, etc., for predicting recurrence of a disease in a patient and a treatment outcome for the patient. The disease recurrence prediction system improves accuracy of prediction and identifies patterns of colon cancer previously undistinguished by the TNM staging system by integrating TNM classification factors with non-TNM classification factors in a prognostic model. The disease recurrence prediction system's pathology framework that integrates clinical histopathological information with quantitative imaging features and molecular biomarker profiles, substantially improves the accuracy of cancer outcome prediction. For example, disease recurrence prediction system's pathology framework can create predictive models for prostate cancer for predicting biochemical recurrence and clinical failure. The disease recurrence prediction system employs imaging algorithms that maintain robustness required for processing large amounts of histopathological images. The performance and robustness of the disease recurrence prediction system is increased by using intrinsic properties of histopathological images of tissues.

The disease recurrence prediction system performs a quantitative image analysis to predict survival of patients. For example, the disease recurrence prediction system performs quantitative image analysis to predict survival of colon cancer patients after tumor removal. The disease recurrence prediction system's quantitative image analysis techniques allow for the description of morphology-color-texture properties of cancer cells and tumor affected regions. In an embodiment, the effect of tumor-node-metastasis (TNM) classification factors and non-TNM classification factors on survival prediction for a patient can be evaluated by the users, for example, medical doctors, pathologists, clinicians, etc., and the disease recurrence prediction system receives the evaluated information from the users. In order to use an entire tissue section of a histopathological image for quantitative image analysis, the disease recurrence prediction system uses low resolution histopathological images instead of conventionally used high resolution histopathological images comprising a pixel resolution of, for example, about 20×, 40×, etc. The disease recurrence prediction system uses low resolution histopathological images because low resolution histopathological images allow an evaluation of entire cancer architecture of a tissue and provide a context for analysis of cancer affected regions in the histopathological images.

The disease recurrence prediction system segments 103 the histopathological images to generate image components of the histopathological images. During segmentation, the disease recurrence prediction system segments background image components from tissue image components of the histopathological images. The disease recurrence prediction system then segments white space image components from stromal-epithelium image components of the tissue image components of the histopathological images. The white space image components represent pericolonic fat present in tissues. The disease recurrence prediction system then segments stromal image components from epithelium image components of the stromal-epithelium image components of the histopathological images.

The disease recurrence prediction system develops and implements a set of imaging algorithms to automatically segment histopathological images, to identify and stratify disease affected regions, for example, cancer affected regions in histopathological tissue images, and to extract quantitative features from histopathological tissue images, for example, colon tissue images of a patient. By implementing the imaging algorithms, the disease recurrence prediction system identifies a subset of cancer affected regions that help pathologists objectively evaluate tumor images. In an embodiment, accuracy of the automated image segmentation implemented by the disease recurrence prediction system can be confirmed by an expert pathologist. The disease recurrence prediction system develops a set of imaging algorithms that identify histopathological factors, which improve the predictive accuracy of survival of patients, for example, early stage cancer patients. The disease recurrence prediction system develops the imaging algorithms comprising unsupervised dissection to automatically segment histopathological images into major histopathological image components and to extract a broad spectrum of quantitative measurements from these histopathological image components.

The disease recurrence prediction system determines 104 disease affected regions in one or more of the image components of the histopathological images, for example, by performing a spatial analysis of the image components of the histopathological images. The spatial analysis comprises performing an iterative expansion of one or more of the image components of the histopathological images. For example, the disease recurrence prediction system performs an iterative expansion of epithelium image components of a histopathological image of a tissue in order to form the disease affected region in the digital image of the tissue.

Consider an example where the disease recurrence prediction system receives a histopathological image of a colon tissue of a patient from a pathologist via the GUI, for determining cancer affected regions in the colon tissue. A cancer affected tissue comprises a heterogeneous image region that includes several tissue image components. The heterogeneous image region of a cancer affected tissue can only be found by spatial analysis of segmented image components of the cancer affected tissue. Hence, in this example, the disease recurrence prediction system performs a spatial analysis of the histopathological image of the colon tissue for determining the cancer affected regions in the tissue image components of the histopathological image.

The disease recurrence prediction system performs the spatial analysis by treating epithelium image components as an anchor in the iterative expansion. The disease recurrence prediction system iteratively expands the epithelium image components by sequentially absorbing other disease related image components, for example, small sized stromal image components and white space image components of the histopathological image of the colon tissue. The small sized stromal image components and the white space image components act as direct neighbors with the epithelium image components, and share a common boundary with the epithelium image components. A stromal image component or a white space image component is absorbed if the stromal image component or the white space image component is located within a rectangular bounding box that contains an expanding epithelium image component. The condition of absorption of the stromal image component or the white space image component defines spatial relationships between the epithelium image components and adjoined image components. The spatial relationships between the epithelium image components and the adjoined image components form cancer affected regions in the histopathological image. The disease recurrence prediction system ends the iterative expansion when a relative area of the cancer affected regions does not change.

The disease recurrence prediction system partitions 105 the determined disease affected regions in the image components of the histopathological images into multiple clusters. In an embodiment, the disease recurrence prediction system partitions the determined disease affected regions in the image components of the histopathological images by performing a texture based segmentation of the image components of the histopathological images. The disease recurrence prediction system performs a sub-segmentation which is a texture based segmentation of the cancer affected regions due to the heterogeneous nature of cancer affected tissues. The disease recurrence prediction system uses a k-means clustering algorithm for performing unsupervised texture based segmentation to partition cancer affected regions in, for example, 4 clusters. The texture of cancer affected regions is represented by frequency vectors from intensity histograms in m×m patches around pixels, where m is equal to 5. The disease recurrence prediction system uses, for example, principal component analysis (PCA) to decrease feature dimensions of one or more image components of the histopathological images required for clustering. The disease recurrence prediction system clusters the cancer affected regions of the histopathological image by allowing, for example, 10 principal components to keep 85% of data variations.

The disease recurrence prediction system quantitates 106 the clusters of the determined disease affected regions in the image components of the histopathological images based on multiple measurement parameters. The measurement parameters comprise, for example, area, perimeter, color, fractal dimensions of region boundaries, texture features, etc. The area measurement parameters comprise, for example, values of absolute areas in pixels, area ratios such as areas of tissue image components relative with respect to cancer affected regions and cancer necrosis regions, ratio of cancer cluster areas, etc. The color measurement parameters comprise, for example, mean and standard deviation values of intensities calculated over region of image components, etc. The disease recurrence prediction system uses a box counting algorithm for calculating the fractal dimensions of boundaries of the cancer affected regions and the necrosis affected regions. The textures measurement parameters comprise, for example, Haralick contrast features, local contrast and entropy, etc. The disease recurrence prediction system extracts measurements from the cancer affected regions and the cancer necrosis regions of the histopathological images based on the measurement parameters.

The disease recurrence prediction system determines 107 one or more key clusters associated with a heterogeneous nature of a disease outcome from the quantitated clusters of the determined disease affected regions. The disease recurrence prediction system performs a statistical analysis of the quantitative information of the clusters to determine the key clusters that are substantially correlated with a disease outcome. The clusters that belong to the determined disease affected regions in the image components of the histopathological images bear different information associated with a prediction of a disease outcome. The disease recurrence prediction system classifies the clusters into clusters that are associated with a disease outcome, clusters that are less associated with a disease outcome, clusters that are not associated with a disease outcome, etc., and determines the key clusters as the clusters that are most correlated with the disease outcome. The disease recurrence prediction system quantitates 108 the determined key clusters based on the measurement parameters.

The disease recurrence prediction system predicts 109 the recurrence of the disease in the patient and the treatment outcome for the patient by statistical modeling of the patient's survival based on the quantitation of the determined key clusters and the patient information. As used herein, the phrase “statistical modeling of survival” refers to generating one or more prognostic models of a patient's survival of a disease based on an analysis of multiple patients' information diagnosed with the same disease. The statistical modeling of survival can predict a biochemical recurrence of a disease in a patient and clinical failure of a treatment plan for the disease. The patient information used for the statistical modeling of survival comprise, for example, clinical information such as a pathological tumor stage, tumor size, or tumor aggressiveness grade such as Gleason score for prostate cancer, depth of tumor invasion, or number of lymph nodes examined, etc.; demographic information associated with a patient such as age, gender, race, sex, etc.; imaging information such as quantitative features extracted from digital histopathological images that represent a tumor and acquired under a microscope, or from a magnetic resonance imaging (MRI) report, a computed tomography (CT) scan, or an ultrasound; biomarker expressions, genetic markers, etc.

In an embodiment, the disease recurrence prediction system performs the statistical modeling of survival of the patient based on the quantitation of the key clusters and the diagnostic criteria at a time instant before the treatment of the patient and/or a time instant after the treatment of the patient. The disease recurrence prediction system generates two types of statistical survival models for a patient. The two types of statistical survival models are, for example, a pre-treatment statistical survival model that is generated at a time of diagnosis and a post treatment statistical survival model that is generated after a treatment plan is implemented for a patient.

In an embodiment, the disease recurrence prediction system generates one or more treatment plans for a patient based on the statistical modeling of survival of the patient. In this embodiment, the disease recurrence prediction system predicts a probability of a disease free survival of the patient within a time period for each of the generated treatment plans. For example, the disease recurrence prediction system provides variable prognoses for early stage JIB colon cancer patients that are evaluated using large clinical set of information associated with the patients. The statistical modeling of survival generated by the disease recurrence prediction system identifies histopathological factors that improve the predictive accuracy of survival of early stage cancer patients. After the disease recurrence prediction system estimates probability of a patient's disease free survival within a time period for several treatment plans, the disease recurrence prediction system displays results for each of the treatment plans and relevant financial information associated with each of the treatment plans. The disease recurrence prediction system specifically tailors optimal scenarios of treatment for each individual patient based on the patient's information. The disease recurrence prediction system generates healthcare information for a user as a result of computer simulation. The generated healthcare information comprises, for example, recommended treatment plans that are available for a patient, probability of disease free survival within a time period of, for example, 5 years, 7 years, 10 years, etc., for each recommended treatment plan, financial cost of the recommended treatment plans, etc.

The disease recurrence prediction system yields an optimal cancer treatment plan for a patient selected out of several possible treatment scenarios, for example, surgery, radiation, chemotherapy, etc., and conditioned on the state of the disease at a time of statistical modeling of survival. The disease recurrence prediction system models and quantitatively estimates outcomes for possible treatment plans that are available for a patient before an actual treatment is applied on the patient. Each treatment plan is associated with a statistical survival model which computes probability of cancer free survival within a certain time period using a patient's information as an input for the disease recurrence prediction system. The disease recurrence prediction system generates a recommendation for a treatment plan for a patient as clinically optimal, when the treatment plan substantially maximizes cancer patients' chances of survival. For example, when the disease recurrence prediction system calculates that the likelihood of a patient's cancer free survival is largest for a treatment plan compared to other treatment plans, then the disease recurrence prediction system generates a recommendation for that treatment plan for the patient. The disease recurrence prediction system uses additional factors, for example, cost of a treatment plan, quality of life after implementation of a treatment plan, etc., for selecting an optimal treatment plan for the patient, when survival estimates for different treatment plans are comparable, for example, when survival estimates for different treatment plans differ from a maximal range within about 10% for all considered treatment plans.

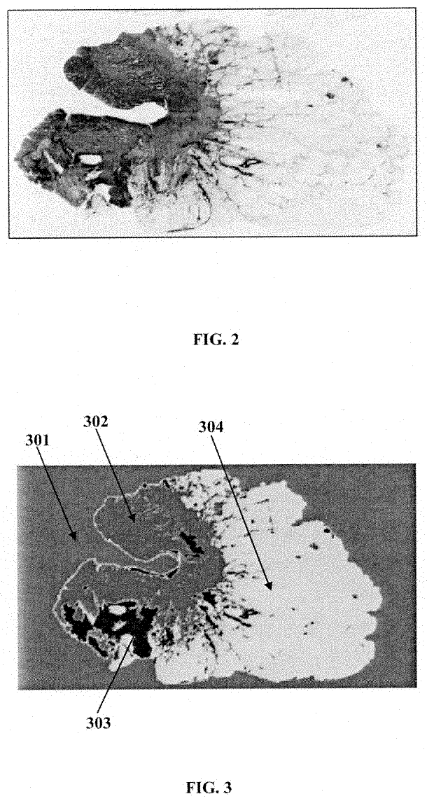

FIG. 2 exemplarily illustrates a histopathological image of a colon tissue. The disease recurrence prediction system receives multiple histopathological images from multiple sources. For example, haematoxylin and eosin (H&E) stained slides of a colon tissue containing deepest invasion from all tumors are first selected and reviewed by an expert pathologist from a cohort of patients detected with stage JIB colon cancer. The H&E stained slides are then scanned and digitized using, for example, ScanScope® digital slide scanner. The histopathological images received from the ScanScope® digital slide scanner are of about 1712×962 pixels are created by the Aperio ImageScope version 11.1 software and saved as images of about 24 bits per pixel in, for example, Tiff format. In an embodiment, the histopathological images received from the Aperio ImageScope version 11.1 software are low resolution snapshots of the H&E stained slides.

FIG. 3 exemplarily illustrates multiple image components of the histopathological image of the colon tissue generated by segmentation of the histopathological image by the disease recurrence prediction system. The disease recurrence prediction system performs a quantitative image analysis to segment the histopathological images into major histopathological components, to identify cancer affected regions in the histopathological images, to extract image measurements in order to develop a statistical survival model for patients, and to cluster cancer affected regions aiming to locate most predictive area. Each histopathological image contains the clusters associated with stroma, necrosis, and lumens. The disease recurrence prediction system recognizes identical clusters on all histopathological images.

The disease recurrence prediction system performs image segmentation as a fully automated and multistep process, which sequentially identifies key components of colon tissues. Equations (1)-(7) below describe color segmentation of a histopathological image of a colon tissue as exemplarily illustrated in FIG. 3. The process of image segmentation begins with segmentation of tissue image components and background image components 301. The disease recurrence prediction system receives the histopathological image as an original red, green, and blue (RGB) histopathological image. The disease recurrence prediction system converts the RGB histopathological image to a gray scale image “I”. The disease recurrence prediction system defines a tissue image component region mask M_{J} using the following equation:

M_{J}={(x,y)|log(1+|∇I(x,y)|>0)} (1)

where |∇I|=\sqrt{∂ₓI²+∂_{y}I²}.

In equation (1), “|∇I|” is an intensity gradient image. The logarithm in equation (1) is used to enhance the process of image segmentation. The disease recurrence prediction system uses cleaning morphological operations to remove small intensity fluctuations in the background image components 301 of the histopathological image.

A tissue is a biological system that consists of numerous elements. Chemical staining colorizes these elements to make them visible for a user, for example, a pathologist. However, colors and shapes of biologically diverse elements are often alike and can only be identified by their spatial location. For example, a white space image component 304 located inside an epithelium image component 302 is an element of cancer. In contrast, a white space image component 304 located outside of the epithelium image component 302 represents fat tissue. Therefore, the disease recurrence prediction system supplements the color based segmentation with spatial analysis of the image components of the histopathological image.

Colon tissue elements have three basic colors, for example, white, red, and purple. Hence, the disease recurrence prediction system performs color based image segmentation for segmenting white space image components 304 and stromal-epithelium image components, and then stromal image components 303 and epithelium image components 302 from the stromal-epithelium image components. Colon tissue area is contained within a tissue mask. A vector c=[r,g,b]^{T} in the red, green, and blue (RGB) space is assigned to each pixel. Directing angle “α” between the vector “c” and R-axes is a good discriminator of white space image components 304 from the stromal-epithelium image components. The value of the directing angle “α” is determined by the following equation:

$$cos α = \frac{r}{\vert c \vert} \qquad ( 2 )$$

where |c|=\sqrt{r²+g²+b²}.

The disease recurrence prediction system defines white space image component's region mask “M_{W}” using the following equation:

M_{W}={(x,y)∈Mⱼ|α(x,y)>α₀} (3)

where α₀=0.8

Stromal-epithelium image component's region mask “M_{δ∈}” are compliments of the white space image component's region mask “M_{W}”, defined by the following equation:

M_{δ∈}={(x,y)∈M_{J}\M_{W}} (4)

The stromal image component 303 is red in color and the epithelium image component 302 is purple in color. The disease recurrence prediction system segments the stromal image components 303 and the epithelium image components 302 using the following equation:

$$cos γ = \frac{b}{\vert c \vert} \qquad ( 5 )$$

The disease recurrence prediction system applies a median filter to reduce sporadic noise. The disease recurrence prediction system replaces values of “cos γ” with indexes “i” after partitioning a range [0,1] of the values of “cos γ” in 10 uniform bins. Each bin represents a sub-range of the values of “cos γ”. Each bin represents a set of pixels with values of cos γ within a same bin. The disease recurrence prediction system assigns each bin an index “i”, where i=1, 2, . . . , 10. The disease recurrence prediction system determines the splitting of the stromal image components 303 and the epithelium image components 302 by using an index “i₀” of a bin with maximum number of pixels “π”. Epithelium image component's region mask “ε” and stromal image component's region mask “δ” are defined by using the following equations (6) and (7):

M_{∈}={(x,y)∈M_{J}\M_{W}|π(x,y)≥i₀} (6)

and

M_{δ}={(x,y)∈M_{J}\M_{W}|π(x,y)<i₀ (7)

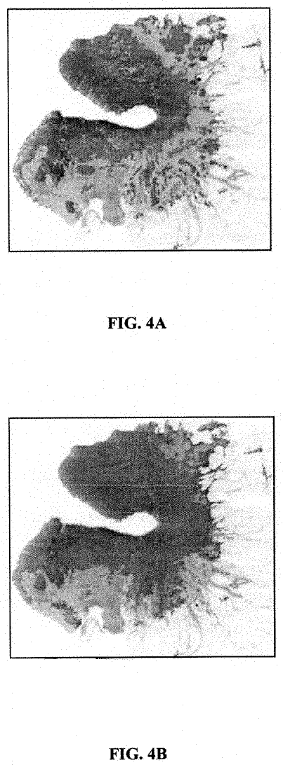

FIG. 4A exemplarily illustrates a histopathological image of a colon tissue with cancer clusters. The disease recurrence prediction system partitions cancer affected regions in image components of the histopathological image of the colon tissue into multiple clusters. The disease recurrence prediction system uses multiple cluster labels, for example, yellow, brown, green, blue, etc. The cluster labels used by different users of the disease recurrence prediction system differ since the clustering of disease affected regions of the histopathological images is done independently by different users. Thus, the disease recurrence prediction system classifies the clusters in all histopathological images to maintain consistency in the classification of the clusters in each of the histopathological images.

FIG. 4B exemplarily illustrates a histopathological image of the colon tissue with relabeled cancer clusters. Mutual proximity of cluster centers is used for the classification. The proximity is described by a matrix “D” that contains pairwise Euclidean distances “dᵢⱼ” and “dⱼᵢ,” between two centers “i” and “j”, where dᵢⱼ=dⱼᵢ. Elements of matrix “D” are normalized such that max_{i,j} dᵢⱼ=1. The most distanced points “i₀” and “j₀”, where dᵢ_{₀}ⱼ_{₀}=1, form two classes for the clusters. The classes are “L₁” and “L₂”. The classes “L₁” and “L₂” have all points within distance d from “i₀” and “j₀”, respectively. Remaining points fall in class “L₃”. Value of “d₀” used for the classification is equal to 0.4. Created classes “Lₖ”, where k=1, 2, 3, allow unified cluster labeling over all histopathological images in a set of histopathological images used by the disease recurrence prediction system for clustering. Re-labeled clusters are exemplarily illustrated in FIG. 4B. The disease recurrence prediction system re-labels yellow-brown clusters to green clusters and green-blue clusters to blue clusters. In an embodiment, the disease recurrence prediction system uses inputs from users registered with the disease recurrence prediction system for cluster evaluation. For example, a pathologist evaluates the clusters and determines that class “L₁” represents necrosis affected regions, class “L₂” represents stromal image components 303 exemplarily illustrated in FIG. 3, and class “L₃” represents lumens.

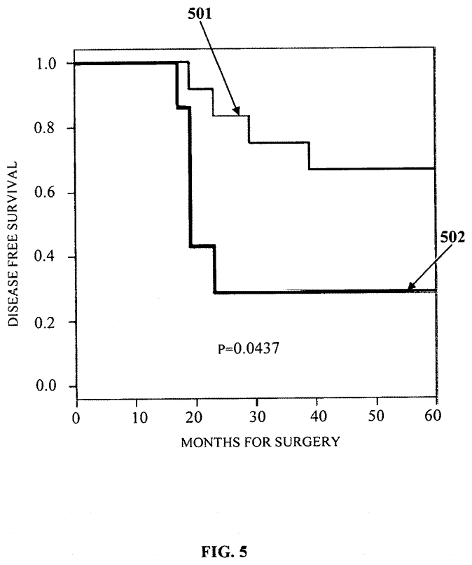

FIG. 5 exemplarily illustrates a graphical representation of an estimation of a disease free survival of multiple early stage colon cancer patients over a period of time, performed by the disease recurrence prediction system. The disease recurrence prediction system generates the graphical representation based on survival difference for early stage colon cancer patients based on multiple predictors, for example, relative necrosis area, Haralick's contrast feature value, etc. Graph line 501 represents patients with low value of relative cancer necrosis area and graph line 502 represents patients with high value of relative cancer necrosis area. The Haralick's contrast feature value indicates poor survival for patients with highest Haralick's contrast feature values. FIG. 5 exemplarily illustrates a poor disease free survival rate of patients with larger, above mean value of cancer necrosis region and higher, above mean value of Haralick's contrast feature in the histopathological images of the patients. A log rank test used to compare survival distributions of patients with low value of relative cancer necrosis area and patients with high value of relative cancer necrosis area showed a probability value “p”=0.0437.

In an example embodiment, the disease recurrence prediction system uses a non-parametric random survival forest methodology, referred herein as a “random survival forest”, that considers all possible interplays among various factors, for example, tumor-node-metastasis (TNM) classification factors and non-TNM classification factors such as clinical factors and histopathological factors. For example, the disease recurrence prediction system uses the random survival forest to identify factors that most accurately predict the survival of early stage colon cancer patients.

The disease recurrence prediction system uses the random survival forest that is an extension of the random forest methodology to right-censored multi-dimensional survival data. Consider an example where the disease recurrence prediction system uses the random survival forest to evaluate prognostic significance of 68 variables including imaging features and clinical components, for example, age, gender, depth of tumor invasion, number of lymph nodes examined, etc. In the random survival forest analysis, the disease recurrence prediction system uses histopathological images of 18 cancer patients who have 9 recurrences of the cancer disease. The random survival forest analysis is used to generate survival curves for each patient. For purposes of illustration, the description refers to use of a non-parametric random survival forest methodology for identifying factors that most accurately predict the survival of early stage colon cancer patients; however the scope of the method and system disclosed herein is not limited to the non-parametric random survival forest methodology but may be extended to include other methodologies for statistical modeling of the patient's survival.

Relative area and Haralick's contrast features of a cancer necrosis region are identified as the most statistically significant predictors of survival for early stage colon cancer patients. In the random survival forest model, increased area of cancer necrosis region relative to the total cancer affected regions and higher value of Haralick's contrast features based on gray level co-occurrence matrix of the cancer necrosis region are associated with poor prognosis. The predictive random survival forest model stratifies patients into low risk groups and high risk groups.

The disease recurrence prediction system taught in the present invention may be used to predict recurrence of other types of cancer in addition to colon cancer. A non-limiting list of cancers of other organs includes lung and pulmonary system cancers, adrenal and lymphatic system cancers, breast cancers, genito-urinary system cancers, mouth, tongue, laryngeal and esophageal cancers, gastrointestinal system cancers, blood cancers, nasopharyngeal system cancers, reproductive system cancers, central nervous system cancers, dermal cancers, and cancers of the kidney, liver, pancreas, and eyes. The disease recurrence prediction system taught in the present invention may be used to predict recurrence of cancers in terminal and non-terminal phases and may be used to predict recurrence of diseases other than cancer.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials, and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention in its aspects.

We claim:

1. A computer implemented method for predicting recurrence of a disease in a patient based on a quantitative image analysis, comprising:
Step 1: providing a clinical decision support application executable by at least one processor configured to predict recurrence of said disease in said patient based on said quantitative image analysis, and clinical patient data;
Step 2: collecting H&E stained slides of colon tissues, scanning said stained slides of said colon tissues, and storing low resolution digitized histopathological images of said stained slides in a database;
Step 3: using low resolution digitized histopathological images to find the entire cancerous or other disease affected regions on digitized histopathological images of stained slides for said quantitative image analysis by clinical decision support application;
Step 4: identifying and segmenting background image components and tissue components of said histopathological image by converting said histopathological image into a grayscale histopathological image by said clinical decision support application;
Step 5: identifying different tissue components of said histopathological images based on their color and textural properties by said clinical decision support application and performing color, textural and boundary based segmentation of said colored histopathological image by:
Step 5a: segmenting white space components from stromal-epithelium tissue components of said histopathological images by said clinical decision support application; and
Step 5b: segmenting stromal tissue components from epithelium tissue components of said stromal-epithelium tissue components of said histopathological images by said clinical decision support application;
Step 6: performing spatial analysis of said segmented tissue components of said histopathological images by said clinical decision support application for automated determining disease affected regions of said colon tissues, wherein said spatial analysis comprises performing iterative expansion of said epithelium tissue components of said histopathological images;
Step 7: partitioning said determined disease affected regions of said colon tissues into a plurality of clusters by said clinical decision support application via texture based sub-segmentation and principal component analysis, wherein said clinical decision support application is configured to classify said clusters using colored cluster labels;
Step 8: assigning labels to obtained said clusters by said clinical decision support application based on mutual proximity of the cluster centers, created said labels allow unified cluster labeling within said disease affected regions over all said segmented histopathological images used by the disease recurrence prediction system;
Step 9: quantitating said clusters of said determined disease affected regions of said colon tissues based on a plurality of factors by said clinical decision support application, wherein said factors comprise area, perimeter, color, fractal dimension of region boundaries, texture features, etc.;
Step 10: predicting recurrence of said disease in said patient by said clinical decision support application via statistical modeling of survival risk of said patient based on said quantitation of said clusters and analytical actions which result in computing survival curves to stratify said patient into a low risk or high risk patient; and
Step 11: predicting probability of disease free survival of said patient within a time period for several possible treatment options by said clinical decision support application based on a plurality of comprehensive data, wherein said clinical decision support application is configured to model and quantitatively estimate likelihood of outcomes for possible treatments that are available for said patient before said optimal treatment plan is applied, and wherein said comprehensive data comprise clinical data, available technology, financial cost, demographic information, imaging, biomarker expression, genetic markers, quality of life during and after treatment, and professional experience and specialty;
Step 12: choosing of optimal treatment option based on said maximal predicted probability of disease free survival of said patient within a time period for several possible treatment options by said clinical decision support application, financial cost, and quality of life for said possible treatment options.

2. A computer based method for choosing an optimal treatment for a disease where several alternative treatments exist, based on predicting probability of disease recurrence or outcome in a patient using relevant quantitative image analysis, comprising:
Step 1: providing a clinical decision support application executable by at least one processor configured to predict recurrence of said disease in said patient based on said quantitative image analysis, and clinical patient data;
Step 2: collecting and storing digital images obtained by microscopic, MRI, CT, or ultrasound imaging of said disease tissues that are used in said alternative treatments of said disease;
Step 3: using low resolution images to find the entire disease affected region or high resolution images for specific small biological elements for said quantitative image analysis by the said clinical decision support application;
Step 4: identifying and segmenting of said digital images to find basic biological tissue elements by said clinical decision support application;
Step 5: performing a spatial analysis of said segmented tissue components of said digital tissue images by said clinical decision support application for automated determining disease affected regions of said disease tissues;
Step 6: partitioning said determined disease-affected regions of said disease tissues into a plurality of clusters by said clinical decision support application via texture based sub-segmentation and principal component analysis, wherein said clinical decision support application is configured to classify said clusters using cluster labels;
Step 7: assigning labels to obtained said clusters by said clinical decision support application based on mutual proximity of the cluster centers, creating said labels allow unified cluster labeling within said disease affected regions over all said segmented digital images used by the disease recurrence prediction system;
Step 8: quantitating said clusters of said determined disease affected regions of said disease tissues based on a plurality of factors by said clinical decision support application, wherein said factors are selected from the group consisting essentially of area, perimeter, color, fractal dimension of region boundaries, and texture features;
Step 9: classifying the clusters by said clinical decision support application into clusters that are associated with the disease outcome, clusters that are less associated with the disease outcome, clusters that are not associated with the disease outcome and determining the key clusters that are most associated with the disease outcome via statistical analysis and/or expert knowledge;
Step 10: developing competitive mathematical models computing probability of disease-free survival within a time period or disease outcome for each considered treatment option of said disease via statistical modeling of survival of said patient based on said quantitation of said the segmented disease affected regions and key clusters, clinical data using statistical analytical tools computing survival curves;
Step 11: predicting probability of disease free survival of said patient within a time period for each possible treatment options by said clinical decision support application based on a plurality of comprehensive data, wherein said clinical decision support application is configured to model and quantitatively estimate likelihood of outcomes for possible treatments that are available for said patient before said optimal treatment plan is applied, and wherein said comprehensive data comprise clinical data, available technology, financial cost, demographic information, imaging, biomarker expression, genetic markers, quality of life during and after treatment, and professional experience and specialty;
Step 12: choosing of optimal treatment option based on said maximal predicted probability of disease free survival of said patient within a time period for each possible treatment options by said clinical decision support application, financial cost, and quality of life for said possible treatment options.

3. The method of claim 2 wherein the said disease is cancer.

4. The method of claim 3 wherein the disease tissue is colon tissue.

5. The method of claim 2 wherein the disease is selected from the group consisting essentially of includes lung and pulmonary system cancers, adrenal and lymphatic system cancers, breast cancers, genito-urinary system cancers, mouth, tongue, laryngeal and esophageal cancers, gastrointestinal system cancers, blood cancers, nasopharyngeal system cancers, reproductive system cancers, central nervous system cancers, dermal cancers, and cancers of the kidney, liver, pancreas, and eyes.

6. The method of claim 2 wherein a web platform for uploading relevant individual patient data (clinical data and said digital tissue images) that are used by said decision support application to compute probability of disease free survival or disease outcome for said each possible treatment, comprising:
Step 1: graphical user interface for said patient data uploading and communicating with the data processing center;
Step 2: representing individual predicting results for said patient for each possible treatment comprising: probability of disease free survival within a time period or disease outcome, potential financial cost, and quality of life after each treatment.
